(19) United States
(12) Patent Application Publication
(10) Pub. No.: US 2005/0100617 A1
(43) Pub. Date: May 12, 2005

(54) Method for improving age-related physiological deficits and increasing longevity

(76) Inventors: Armand Malnoe, (US); Sylvie Pridmore-Merten, (US)

Correspondence Address: WINSTON & STRAWN;PATENT DEPARTMENT, 1400 L STREET, N.W., WASHINGTON, DC, 20005-3502, US

(21) Appl. No.: 11/011,223

(22) Filed: Dec. 13, 2004

Related U.S. Application Data
(63) Continuation-in-part of application No. 10/656,955, filed on Sep. 5, 2003, now abandoned.
Application No. 10/656,955 is a continuation of application No. PCT/EP02/02862, filed on Mar. 7, 2002.

(30) Foreign Application Priority Data
Mar. 9, 2001 (EM) 01200871.0

(51) Int. Cl. A61K 35/78; A61K 38/05; A61K 31/198; A61K 31/385; A61K 31/355; A61K 31/375

(52) U.S. Cl. 424/728; 514/18; 424/757; 424/752; 424/729; 424/766; 424/776; 514/565; 514/690; 514/562; 514/458; 514/440; 514/763; 514/474; 514/46

(57) ABSTRACT

A method for mimicking the effects of caloric restriction by administration of a food substrate having carnitine or a carnitine derivative and an antioxidant. The food substrate is capable of modulating gene expression in a way similar to caloric restriction.

### CROSS-REFERENCE TO RELATED APPLICATION

The present application is a continuation-in-part of U.S. patent application Ser. No. 10/656,955, filed Sep. 5, 2003, which is a continuation of International application PCT/EP02/02862 filed Mar. 7, 2002, the entire content each are expressly incorporated herein by reference thereto.

### TECHNICAL FIELD

Generally, this invention relates to a method for improving age-related physiological deficits and extending life span in mammals as well as improving the condition of elderly mammals. In particular, the invention relates to a method for reducing mitochondrial dysfunction occurring in mammals during aging. Additionally, the method of the invention mimics the effects of caloric restriction on gene expression.

### BACKGROUND OF THE INVENTION

Scientists have found that substantially reducing an organism's caloric intake increases longevity in mammals. Caloric restriction also known as “undernutrition without malnutrition” refers to a daily diet having about 30 to 40% fewer calories than the typical daily diet, but which contains the required nutrients and vitamins to support life.

Research has shown that caloric restriction extends both the maximal and the average life span of mice. In addition, preliminary studies suggest that calorie-restricted monkeys are healthier and tend to live longer than their freely fed counterparts. Mattison J A, Lane M A, Roth G S, Ingram D K. Calorie restriction in rhesus monkeys. Exp Gerontol 2003; 38: 35-46, the content of which is incorporated herein by reference.

In addition to increasing an organism's life span, caloric restriction plays a role in preventing or delaying many age-associated diseases and conditions, such as heart disease, dementia, and cancer. It has been found that caloric restriction not only slows the effects of aging on the nervous system, but studies suggest that it boosts the immune system and delays the onset of certain age-related cancers. Barzilai N, Gupta G. Revisiting the role of fat mass in the life extension induced by caloric restriction. J Gerontol A Biol Sci Med Sci 1999; 54: B89-96, the content of which is incorporated herein by reference.

Mitochondria are cellular organelles often referred to as the “powerhouses” of the cell because they are the sites for cellular respiration or energy production in the cell. Indeed, mitochondria generate most of the energy of the cell primarily through oxidative phosphorylation, a complex process that uses electrons generated through oxidation of glucose and fatty acids to generate ATP.

Aging mitochondria suffer from impaired function, which is associated with a variety of functional deficits (both physical and cognitive) and also the development of degenerative diseases. Proteins of the mitochondria oxidative phosphorylation complex have been shown to be impaired upon aging, which leads to a higher production of reactive oxygen species (ROS) and a decrease in efficiency of energy production. Free radicals produced by aerobic respiration cause cumulative oxidative damages resulting in aging and cell death. The biggest impact of age-related increase in ROS appears to be on on somatic tissues composed of post-mitotic non-replicative cells including muscles, e.g., cardiac and skeletal, and nervous tissues, e.g., brain, retinal pigment epithelium.

Numerous age-related changes have been reported in mitochondria. For example, oxidative damage to mitochondria DNA (mt DNA) increases with aging (Beckman K B, Ames B N (1999) Mutat Res. 424 (1-2):51-8), the content of which is incorporated herein by reference, along with the oxidation of glutathione (GSH) a major intracellular antioxidant system, which plays an important role in protection against age-related mt DNA oxidative damage. A substantial increase in protein oxidation is also observed upon aging. Stadtman E R. (1992), Science 257 (5074):1220-4), the content of which is incorporated herein by reference. Age-related increase in the amount of long chain polyunsaturated fatty acids has been linked to the high peroxidizability of the mitochondria lipids upon aging. This is well illustrated by the change in the composition of cardiolipin, a phospholipid found principally in mitochondria, which fatty acid composition tends to shift towards a more unsaturated state with substitution of 18:2 acyl chains with the more peroxidizable 22:4 and 22:5 upon aging. Laganiere S, Yu B P (1993), Gerontology 39 (1):7-18, the content of which is incorporated herein by reference. The mitochondria content in cardiolipin has also been reported to decrease with age. Cardiolipin interacts with many components of the mitochondria inner membrane such as Cytochrome oxidase, transporters/translocators (ADP/ATP, phosphate, pyruvate, carnitine, etc) and plays an active role in their activity (Hoch F L. (1992) Biochim Biophys Acta. 1113 (1):71-133; Paradies G, Ruggiero F M. (1990) Biochimn Biophys Acta. 1016(2):207-12), each of the contents of which are incorporated herein by reference.

Energy metabolism depends upon the transport of metabolites such as pyruvate across the mitochondria inner membrane. Pyruvate transport is carrier-mediated (Hoch F L. (1988) Prog Lipid Res. 27 (3):199-270, the content of which is incorporated herein by reference ) and a requirement for cardiolipin has been demonstrated for optimal pyruvate translocase activity (Paradies G, Ruggiero F M. (1990) Biochim Biophys Acta. 1016 (2):207-12, the content of which is incorporated herein by reference). Other modifications such as decrease in mitochondria membrane potential and morphological changes e.g., swelling, altered cristae, matrix vacuolisation, are associated with chronic oxidative stress and aging.

Caloric restriction has been observed to retard and even reverse oxidative damage in aging animals. Lass A, Sohal B H, Weindruch R, Forster M J, Sohal R S. Importantly, caloric restriction has been found to prevent age-associated accrual of oxidative damage to mouse skeletal muscle mitochondria. Free Radic Biol Med 1998; 25: 1089-97, the content of which is incorporated herein by reference.

Additionally, it has been found that long-term caloric restriction initiated before mid-life, retards aging and has multiple effects on the metabolism of the cell. Indeed, caloric restriction decreases oxidative damage to DNA, proteins and lipids in rodents (Shigenaga M K, Ames B N. (1994) in: Natural Antioxidants in Human Health and Disease, B. Frei editor, Academic Press, New York. pp 63-106, the content of which is incorporated by reference, increases motor activity in rodents, reduces fiber loss and the age-related accumulation of dysfunctional fibers. Aspnes L E et al. (1997) FASEB J. 11 (7):573-81, the content of which is incorporated herein by reference.

Although there are many advantages to caloric restriction, the drawbacks of such a diet is both unpractical and not well perceived. Severe caloric restriction can produces weight loss to the point that the subject appears unhealthy. Followers of extreme calorie-restricted diets are generally cold and hungry. The loss of body fat causes a loss in padding and cushioning of the bones. Sitting and walking can be painful due to the pressure on the bones. Taubes G. The famine of youth. Scientific American Presents 2000; 11: 44-9, the content of which is incorporated herein by reference. Thus, the drawbacks of the caloric restriction, despite the founded advantages, causes little compliance.

Therefore, there is a need for method and/or composition that mimics the effects of caloric restriction without requiring subjects to drastically reduce their calorie intake and risk potentially dangerous side effects.

### SUMMARY OF THE INVENTION

It has surprisingly been found that the effects of caloric restriction (CR) on gene expression and the advantages resulting from such can be mimicked by nutritional intervention. It is now possible to modulate gene expression of target without drastically reducing caloric intake and suffering from the variety of discomfort associated with CR. The present method advantageously prevent the age-related changes and improves at least one of skeletal and cardiac muscle function, vascular function, cognitive function, vision, hearing olfaction, skin and coat quality, bone and joint health, renal health, digestion, immune function, insulin sensitivity, inflammatory processes, and longevity in mammals.

In accordance with one aspect of the invention, a method is provided for mimicking the effects of caloric restriction on gene expression of target genes. The phrase “mimic” or “mimicking” the effect of caloric restriction refers to the similarity of the gene expression changes induced by the carnitine and antioxidant combination, as well as the physiological, biological and behavioral similarities between the present invention to caloric restriction. The method includes administering to a mammal an effective amount of carnitine in combination with at least one antioxidant. It has surprisingly been found that daily administration of the camitine and antioxidants effects target genes in a way strikingly similar to a caloric restriction. Target genes can be genes which activity are shown to be directly affected during aging (direct effect) or genes for which activity prevents age-related changes to occur (indirect effect). One obvious advantage of the present method is that the caloric intake of the mammal need not be drastically reduced. Thus, the suffering of hunger and the uncomfortable consequences of drastic body fat loss from a drastically reduced calorie diet such as caloric restriction is not necessary to obtain the benefits of the diet. In this respect, the method includes modulating gene expression of a target gene without restricting caloric intake.

The genes targeted are preferably, but not exclusively, involved in any of the following, apoptosis, energy production, chromatin organization, mitochondria biogenesis, protein and lipid metabolism, or free radical production, free radical detoxification or modulators of inflammatory and immune response.

As mentioned above, the aging mitochondria and oxidative damage has been found to be largely responsible for a variety of functional deficits and the development of degenerative diseases. Advantageously, the method is capable of reversing or retarding oxidative damage to the mitochondria.

The carnitine is administered to the mammal in an amount of at least 1 mg per kg of body weight per day. The antioxidant can be one or more of thiol, lipoic acid, cysteine, cystine, methionine, S-adenosyl-methionine, taurine, glutathione, vitamin C, vitamin E, tocopherols and tocotrienols, carotenoids, carotenes, lycopene, lutein, zeaxanthine, ubiquinones, tea catechins, coffee extracts, ginkgo biloba extracts, grape or grape seed extracts, spice extracts, soy extracts, containing isoflavones, phytoestrogens ursodeoxycholic acid, ursolic acid, ginseng, or gingenosides, which is administered in an amount of at least 0.025 mg per kg of body weight per day.

The carnitine and the antioxidants may be administered to the mammal in a food substrate such as a nutritionally complete food or a food supplement.

In another aspect of the invention, a method is provided for reducing mitochondrial dysfunction occurring in a mammal during aging comprising modulating gene expression of a target gene by administering to a mammal carnitine in combination with at least one molecule that stimulates energy metabolism. In a preferred embodiment, the molecule that stimulates energy metabolism is any nutrient improving energy production in mitochondria, such as creatine, fatty acids (mono and polyunsaturated, particularly omega-3 fatty acids), cardiolipin, nicotinamide, carbohydrate and natural sources thereof, for example. The combination may further include an antioxidant, such as but not limited to lipoic acid. Advantageously, it has been found that it is in fact possible to target mitochondria function through dietary intervention and have an impact on genes linked to energy metabolism and longevity.

In one embodiment, a method is provided for delaying mitochondria dysfunction occurring in a mammal during aging, which method comprises administering to a mammal in need of or desirous of such treatment a combination that is able to mimic the effects of caloric restriction on gene expression, the combination containing (a) a carnitine compound, and (b) at least one antioxidant in an amount effective to reduce or prevent oxidative damage resulting from disruption of ATP/ADP or NAD+/NADH homeostasis due to increased substrate availability or utilization in aged mitochondria, and being administered in an amount effective to modulate or regulate expression of genes linked to energy metabolism.

As mentioned, the antioxidant aims to prevent or at least reduce oxidative damage that can result from the disruption of the ATP/ADP and/or NAD+/NADH homeostasis due to the increased substrate availability/utilization in the aged mitochondria. Among antioxidants: sources of thiols, compounds that decrease protein oxidation and compounds that upregulate cell antioxidant defenses are preferably used. The term “antioxidant” as used herein refers to any substance capable of inhibiting oxidation. Antioxidants protect key cell components by neutralizing the damaging effects of “free radicals,” natural byproducts of cell metabolism. Free radicals form when oxygen is metabolized, or burned by the body. They travel through cells, disrupting the structure of other molecules, causing cellular damage. It is well documented that such cell damage is believed to contribute to aging and various health problems.

The method may include administering the molecule that stimulates energy metabolism and the at least one antioxidant in a food substrate. The food substrate may be administered to the mammal daily. In this regard, the food substrate may be a nutritionally complete food or a food supplement.

Advantageously, the method of the present invention is capable of retarding or reversing age associated oxidative damage in mammals. Accordingly, the present invention can prevent or delay mitochondrial dysfunctions associated with aging by modulating and/or regulating expression of genes linked to energy metabolism. The method also provides multiple benefits by improving age-related functional deficits e.g. in skeletal and cardiac muscle function, vascular function, cognitive function, vision, hearing, olfaction, skin and coat quality, bone and joint health, renal health, gut function, immune function, insulin sensitivity, inflammatory processes, cancer incidence and ultimately increasing longevity in mammals. In a further aspect, this invention provides a method to prevent or restore age-related functional deficits in mammals, comprising administering to the mammal, a food composition comprising a combination capable of mimicking the effects of caloric restriction on gene expression.

### BRIEF DESCRIPTION OF THE DRAWINGS

FIG. 1 is a graph illustrating the effects of short-term caloric restriction and the experimental diets on gene expression of young mice;

FIG. 2 is a graph illustrating a comparison of long term treatment of a diet comprising carnitine and antioxidant with long term caloric restriction in old mice; and

FIG. 3 is a graph illustrating the effects of long term caloric restriction and the experimental diets on gene expression of old mice.

### DETAILED DESCRIPTION OF THE PREFERRED EMBODIMENTS

In accordance with the invention is a method for mimicking the effects of caloric restriction on gene expression comprising administering to a mammal an effective amount of carnitine and at least one antioxidant. The carnitine and antioxidant is preferably administered in a food substrate comprising an edible substrate and a combination comprising the carnitine and the antioxidants. The food substrate may be a nutritionally complete food substrate or a food supplement.

In accordance with a further aspect of the invention, the method includes modulating gene expression of a target gene without restricting caloric intake. It has been found that the administration of the camitine and the antioxidants of the invention on mammals modulates gene expression of target genes in a strikingly similar fashion as does a mammal on caloric restriction.

Preferably, but not exclusively, the target genes are those genes involved in (1) energy production: glycolysis, gluconeogenesis, oxidative phosphorylation , β-oxidation and tri-caboxylic acid cycle (2) mitochondria biogenesis, proteins synthesis (3) proteases (neutral alkaline protease) (4) ROS production and detoxification (5) modulators of inflammatory and immune response, (6) apoptosis.

As example of target genes the following, non-exhaustive, genes list includes genes involved in:
ATP generation (ATP synthase . . . ),
Glycolysis (lactate dehydrogenase, pyruvate kinase, hexokinase 2, pyruvate kinase, enolase, phosphoglycerate kinase, dihydrolipoamide dehydrogenase, succinate-Coenzyme A ligase, ADP-forming, beta subunit),
Gluconeogenesis (pyruvate carboxylase),
Electron transport (NADH dehydrogenase (ubiquinone), cytochrome c oxidase, acyl-Coenzyme A oxidase, cytochrome c oxidase subunit VIIb, cytochrome c oxidase subunit IV isoform, glutaryl-Coenzyme A dehydrogenase, ubiquinol-cytochrome c reductase, electron transferring flavoprotein, cytochrome c, MYB binding protein, nicotinamide nucleotide transhydrogenase.
β-oxidation (carnitine carrier, Carnitine transferase . . . )
Inflammatory and immune response (interleukin 5, histocompatibility 2, interleukin 9, tumor necrosis factor (ligand) superfamily, member 7, guanylate nucleotide binding protein 2, toll interacting protein, chemokine (C—C motif) ligand 2, chemokine (C—C motif) ligand 8, chemokine (C—X—C motif) ligand 2.),
Mitochondria biogenesis (HSP70 . . . ),
Fatty acid and lipid metabolism (fatty acid synthase, stearoyl-CoA desaturase, 1-acylglycerol-3-phosphate O-acyltransferase 3, apolipoprotein A-V, apolipoprotein E, enoyl coenzyme A hydratase 1, L-3-hydroxyacyl-Coenzyme A dehydrogenase, short chain, lipoprotein lipase, lysophospholipase 1, lysophospholipase 2, monoacylglycerol O-acyltransferase, NADH dehydrogenase (ubiquinone), phospholipase A2.
Protein turnover (proteasome subunit, ribosomal proteins, . . . ),
Stress response (catalase,superoxide dismutase 1, soluble phospholipase A2, group IB, advillin)
Apoptosis: HLA-B-associated transcript 3, BCL2/adenovirus E1B interacting protein 1, Fas-associated factor 1, ring finger protein 7, cullin 1, BH3 interacting domain death agonist, CCAAT/enhancer binding protein (C/EBP).
Transcription regulation: thyroid hormone receptor, retinoid X receptor alpha

The carnitine is preferably L-carnitine, the acetyl-derivative of L-carnitine (ALCAR) or the propionyl L-carnitine, and is preferably administered an amount of at least 1 mg per kg of body weight per day, more preferably from 1 mg to 1 g per kg of body weight per day.

The antioxidants are compounds that decrease protein oxidation (e.g. prevent formation of protein carbonyls). They may be sources of thiols (e.g. Lipoic acid, cysteine, cystine, methionine, S-adenosyl-methionine, taurine, glutathione and natural sources thereof), or compounds that upregulate their biosynthesis in vivo, for example.

The antioxidant according to the invention may be used either alone or in association with other antioxidants such as vitamin C, vitamin E (tocopherols and tocotrienols), carotenoids (carotenes, lycopene, lutein, zeaxanthine . . . ) ubiquinones (e.g.CoQ10), tea catechins (e.g. epigallocatechin gallate), coffee extracts containing polyphenols and/or diterpenes (e.g. kawheol and cafestol), ginkgo biloba extracts, grape or grape seed extracts rich in proanthocyanidins, spice extracts (e.g. rosemary), soy extracts containing isoflavones and related phytoestrogens and other sources of flavonoids with antioxidant activity, compounds that upregulate cell antioxidant defense (e.g. ursodeoxycholic acid for increased glutathione S-transferase, ursolic acid for increased catalase, ginseng and gingenosides for increase superoxide dismutase and natural sources thereof i.e. herbal medicines).

Preferably, the amount of the antioxidant is of at least 0.025 mg per kg of body weight per day, more preferably from 0.025 mg to 250mg per kg of body weight per day.

The present method improves mitochondrial function, and is capable of retarding or reversing age-associated oxidative damage to the mitochondria. Advantageously, the method provides multiple benefits including improving at least one of skeletal and cardiac muscle function, vascular function, cognitive function, vision, hearing olfaction, skin and coat quality, bone and joint health, renal health, digestion, immune function, insulin sensitivity, inflammatory processes, and longevity in mammals.

In accordance with another aspect of the invention a method is provided for reducing mitochondrial dysfunction occurring in a mammal during aging. The method includes modulating gene expression of the target gene by administering to a mammal a combination comprising at least one molecule that stimulates energy metabolism, and at least one antioxidant.

In one embodiment, the mammal is administered a food composition containing the combination of at least one molecule that stimulates energy metabolism of the cell and at least one antioxidant and the food composition is capable of mimicking the effects of caloric restriction on gene expression.

The molecule that stimulates energy metabolism of the cell and in particular the energy metabolism of the mitochondria may be L-carnitine, creatine, fatty acids (mono or polyunsaturated fatty acids, particularly omega-3 fatty acids), cardiolipin, nicotinamide, carbohydrate and natural sources thereof, for example. The antioxidant has been described above.

Preferably, the amount of the food composition to be consumed by the mammal to obtain a beneficial effect will depend upon its size, its type, and its age. However an amount of said molecule of at least 1 mg per kg of body weight per day and an amount of the antioxidant of at least 0.025 mg per kg of body weight per day, would usually be adequate.

The composition may be administered to the mammal as a supplement to the normal diet or as a component of a nutritionally complete food. It is preferred to prepare a nutritionally complete food. Accordingly, with respect to another object of the present invention, a food composition intended to prevent or restore age-related functional deficits in mammals by reversing age-related gene expression alterations, which comprises a combination being able to mimic the effects of caloric restriction on gene expression, said combination containing at least one molecule that stimulates energy metabolism of the cell and at least one antioxidant. The food composition comprising a molecule capable of stimulating energy metabolism of a cell and a combination of antioxidants. In a preferred embodiment, the molecule stimulates in particular energy metabolism of the mitochondria.

Indeed, it has been surprisingly found that the effects of caloric restriction on gene expression can be mimicked by nutritional interventions that do not limit calorie intake but result in improved mitochondria function.

In one embodiment, a nutritionally complete pet food can be prepared. The nutritionally complete pet food may be in any suitable form; for example in dried form, semi-moist form or wet form; it may be a chilled or shelf stable pet food product. These pet foods may be produced as is conventional. Apart from the combination according to the invention, these pet foods may include any one or more of a carbohydrate source, a protein source and lipid source.

Any suitable carbohydrate source may be used. Preferably the carbohydrate source is provided in the form of grains, flours and starches. For example, the carbohydrate source may be rice, barley, sorghum, millet, oat, corn meal or wheat flour. Simple sugars such as sucrose, glucose and corn syrups may also be used. The amount of carbohydrate provided by the carbohydrate source may be selected as desired. For example, the pet food may contain up to about 60% by weight of carbohydrate.

Suitable protein sources may be selected from any suitable animal or vegetable protein source; for example muscular or skeletal meat, meat and bone meal, poultry meal, fish meal, milk proteins, corn gluten, wheat gluten, soy flour, soy protein concentrates, soy protein isolates, egg proteins, whey, casein, gluten, and the like. For elderly animals, it is preferred for the protein source to contain a high quality animal protein. The amount of protein provided by the protein source may be selected as desired. For example, the pet food may contain about 12% to about 70% by weight of protein on a dry basis.

The pet food may contain a fat source. Any suitable fat source may be used both animal fats and vegetable fats. Preferably the fat source is an animal fat source such as tallow. Vegetable oils such as corn oil, sunflower oil, safflower oil, rape seed oil, soy bean oil, olive oil and other oils rich in monounsaturated and polyunsaturated fatty acids, may also be used. In addition to essential fatty acids (linoleic and alpha-linoleic acid) the fat source may include long chain fatty acids. Suitable long chain fatty acids include, gamma linoleic acid, stearidonic acid, arachidonic acid, eicosapentanoic acid, and docosahexanoic acid. Fish oils are a suitable source of eicosapentanoic acids and docosahexanoic acid. Borage oil, blackcurrent seed oil and evening primrose oil are suitable sources of gamma linoleic acid. Rapeseed oil, soybean oil, linseed oil and walnut oil are suitable sources of alpha-linoleic acid. Safflower oils, sunflower oils, corn oils and soybean oils are suitable sources of linoleic acid. Olive oil, rapeseed oil (canola) high oleic sunflower and safflower, peanut oil, rice bran oil are suitable sources of monounsaturated fatty acids. The amount of fat provided by the fat source may be selected as desired. For example, the pet food may contain about 5% to about 40% by weight of fat on a dry basis. Preferably, the pet food has a relatively reduced amount of fat.

The pet food may contain other active agents such as long chain fatty acids. Suitable long chain fatty acids include alpha-linoleic acid, gamma linoleic acid, linoleic acid, eicosapentanoic acid, and docosahexanoic acid. Fish oils are a suitable source of eicosapentanoic acids and docosahexanoic acid. Borage oil, blackcurrent seed oil and evening primrose oil are suitable sources of gamma linoleic acid. Safflower oils, sunflower oils, corn oils and soybean oils are suitable sources of linoleic acid.

The choice of the carbohydrate, protein and lipid sources is not critical and will be selected based upon nutritional needs of the animal, palatability considerations, and the type of product produced. Further, various other ingredients, for example, sugar, salt, spices, seasonings, vitamins, minerals, flavoring agents, gums, prebiotics and probiotic micro-organisms may also be incorporated into the pet food as desired

The prebiotics may be provided in any suitable form. For example, the prebiotic may be provided in the form of plant material, which contains the prebiotic. Suitable plant materials include asparagus, artichokes, onions, wheat, yacon or chicory, or residues of these plant materials. Alternatively, the prebiotic may be provided as an inulin extract or its hydrolysis products commonly known as fructooligosaccharides, galacto-oligosaccarides, xylo-oligosaccharides or oligo derivatives of starch. Extracts from chicory are particularly suitable. The maximum level of prebiotic in the pet food is preferably about 20% by weight; especially about 10% by weight. For example, the prebiotic may comprise about 0.1% to about 5% by weight of the pet food. For pet foods which use chicory as the prebiotic, the chicory may be included to comprise about 0.5% to about 10% by weight of the feed mixture; more preferably about 1% to about 5% by weight.

The probiotic microorganism may be selected from one or more microorganisms suitable for animal consumption and which is able to improve the microbial balance in the intestine. Examples of suitable probiotic micro-organisms include yeast such as Saccharonyces, Debaroinyces, Candida, Pichia and Torulopsis, moulds such as Aspergillus, Rhizopus, Mucor, and Penicillium and Torulopsis and bacteria such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus and Lactobacillus. Specific examples of suitable probiotic micro-organisms are: Saccharomyces cereviseae, Bacillus coagulans, Bacillus licheniformis, Bacillus subtilis, Bifidobacterium bifiduin, Bifidobacterium infantis, Bifidobacterium longum, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbruckii subsp. lactis, Lactobacillus farciminus, Lactobacillus gasseri, Lactobacillus helveticus, Lactobacillus johnsonii, Lactobacillus reuteri, Lactobacillus rhamnosus (Lactobacillus GG), Lactobacillus sake, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus, and Staphylococcus xylosus. The probiotic micro-organisms may be in powdered, dried form; especially in spore form for micro-organisms which form spores. Further, if desired, the probiotic micro-organism may be encapsulated to further increase the probability of survival; for example in a sugar matrix, fat matrix or polysaccharide matrix. If a probiotic micro-organism is used, the pet food preferably contains about 10⁴ to about 10¹⁰ cells of the probiotic micro-organism per gram of the pet food; more preferably about 10⁶ to about 10⁸ cells of the probiotic micro-organism per gram. The pet food may contain about 0.5% to about 20% by weight of the mixture of the probiotic micro-organism; preferably about 1% to about 6% by weight; for example about 3% to about 6% by weight.

For elderly pets, the pet food preferably contains proportionally less fat than pet foods for younger pets. Further, the starch sources may include one or more of oat, rice, barley, wheat and corn.

For dried pet foods a suitable process is extrusion cooking, although baking and other suitable processes may be used. When extrusion cooked, the dried pet food is usually provided in the form of a kibble. If a prebiotic is used, the prebiotic may be admixed with the other ingredients of the dried pet food prior to processing. A suitable process is described in European patent application No 0850569;. If a probiotic micro-organism is used, the organism is best coated onto or filled into the dried pet food. A suitable process is described in European patent application No 0862863.

For wet foods, the processes described in U.S. Pat. Nos. 4,781,939 and 5,132,137 may be used to produce simulated meat products. Other procedures for producing chunk type products may also be used; for example cooking in a steam oven. Alternatively, loaf type products may be produced by emulsifying a suitable meat material to produce a meat emulsion, adding a suitable gelling agent, and heating the meat emulsion prior to filling into cans or other containers.

In another embodiment, a food composition for human consumption is prepared. This composition may be a nutritional complete formula, a dairy product, a chilled or shelf stable beverage, soup, a dietary supplement, a meal replacement, and a nutritional bar or a confectionery.

Apart from the combination according to the invention, the nutritional formula may comprise a source of protein. Dietary proteins are preferably used as a source of protein. The dietary proteins may be any suitable dietary protein; for example animal proteins (such as milk proteins, meat proteins and egg proteins); vegetable proteins (such as soy protein, wheat protein, rice protein, and pea protein); mixtures of free amino acids; or combinations thereof. Milk proteins such as casein, whey proteins and soy proteins are particularly preferred. The composition may also contain a source of carbohydrates and a source of fat.

If the nutritional formula includes a fat source, the fat source preferably provides about 5% to about 55% of the energy of the nutritional formula; for example about 20% to about 50% of the energy. The lipids making up the fat source may be any suitable fat or fat mixtures. Vegetable fats are particularly suitable; for example soy oil, palm oil, coconut oil, safflower oil, sunflower oil, corn oil, canola oil, lecithins, and the like. Animal fats such as milk fats may also be added if desired.

A source of carbohydrate may be added to the nutritional formula. It preferably provides about 40% to about 80% of the energy of the nutritional composition. Any suitable carbohydrates may be used, for example sucrose, lactose, glucose, fructose, corn syrup solids, and maltodextrins, and mixtures thereof. Dietary fiber may also be added if desired. If used, it preferably comprises up to about 5% of the energy of the nutritional formula. The dietary fiber may be from any suitable origin, including for example soy, pea, oat, pectin, guar gum, gum arabic, and fructooligosaccharides. Suitable vitamins and minerals may be included in the nutritional formula in an amount to meet the appropriate guidelines.

One or more food grade emulsifiers may be incorporated into the nutritional formula if desired; for example diacetyl tartaric acid esters of mono- and di-glycerides, lecithin and mono- and di-glycerides. Similarly suitable salts and stabilizers may be included.

The nutritional formula intended improving or preventing age-related functional deficits is preferably enterally administrable; for example in the form of a powder, a liquid concentrate, or a ready-to-drink beverage. If it is desired to produce a powdered nutritional formula, the homogenized mixture is transferred to a suitable drying apparatus such as a spray drier or freeze drier and converted to powder.

In another embodiment, a usual food product may be enriched with the combination according to the present invention. For example, a fermented milk, a yogurt, a fresh cheese, a renneted milk, a confectionery bar, breakfast cereal flakes or bars, drinks, milk powders, soy-based products, non-milk fermented products or nutritional supplements for clinical nutrition. Then, the amount of the molecule that stimulates energy metabolism is preferably of at least about 50 ppm by weight and the antioxidant is preferably of at least 10 ppm by weight.

According to another aspect, this invention relates to the preparation of a composition intended to prevent or restore age-related functional deficits in mammals. This preparation includes the use of a combination that is able to mimic the effects of caloric restriction on gene expression, which combination comprises at least one molecule that stimulates energy metabolism of the cell and at least one antioxidant. The molecule and antioxidant have been described above.

Tables 1 and 2 below, illustrate the effects of the present invention after short treatments on gene expression of target genes in young mammals. Also shown in table 3 are the effects of long treatment of the present method on gene expression of target genes in old mammals.

Tables 1 and 2, entitled Gene Selected As Significantly Modulated By Caloric Restriction And Supplementation With L-Carnitine And A Cocktail Of Antioxidants In Skeletal Muscle of Young Mice After Three Month Treatment, is a comparison of the changes of gene expression, which are induced by each diets of the study when compared to the control diet (diet A). The experimental diets include diet B, (caloric restriction), diet C (antioxidant alone), diet D (L-carnitine and antioxidants) and diet F (L-camitine alone).

Tables 1 and 2 shows the regulation of expression of target genes when expressed as fold changes. For each target gene, the fold change is calculated as follows: [Expression obtained with the experimental diet] divided by [Expression obtained with control diet]. Consequently, fold changes of all target genes are equal to 1 (one) for the control diet. Thus, fold changes refers to the modulation of expression of a given target gene in muscle tissue after 3 months feeding with an experimental diet, for example, diets B, C, D, or F, when compared to the expression of the same gene in muscle tissue of the control group (diet A).

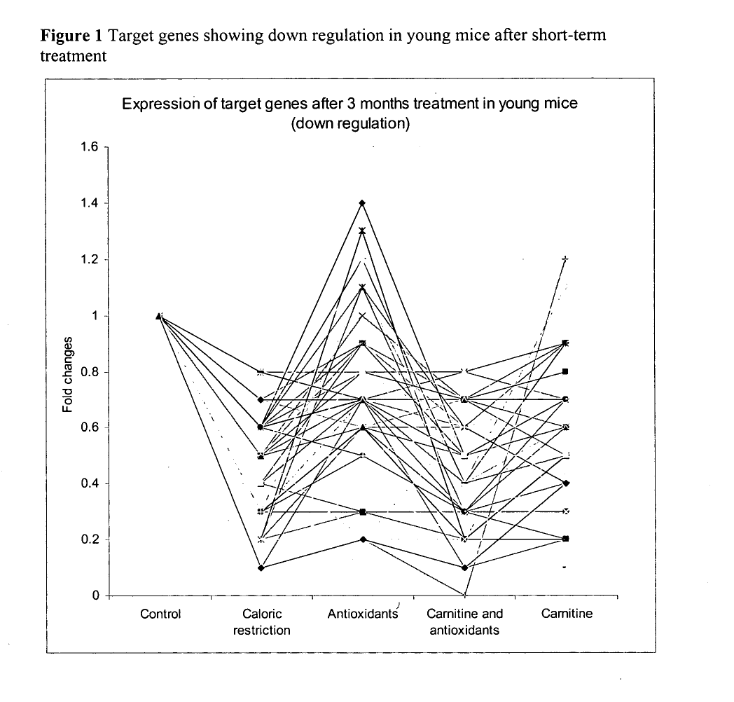

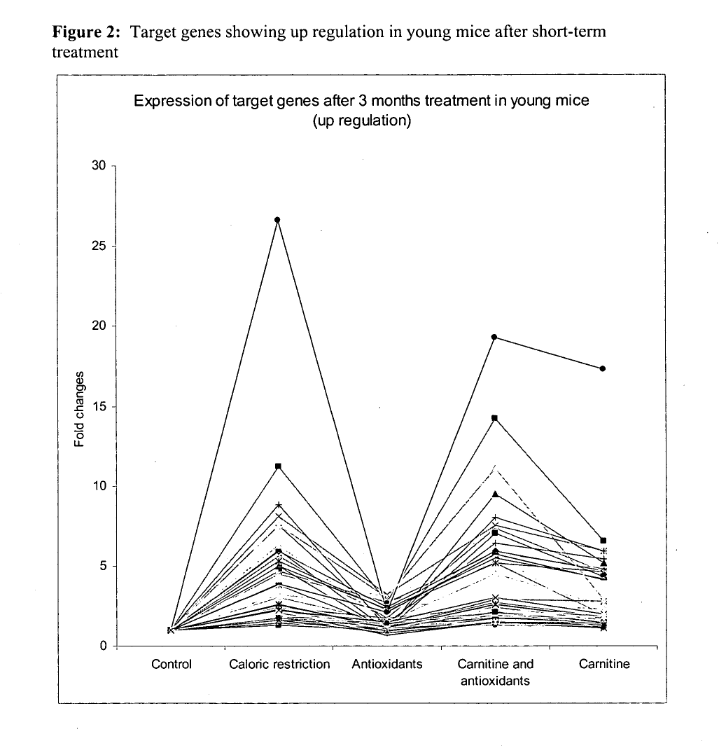

As shown, caloric restriction induces changes in the gene expression of the target genes. A comparison of the effects of the caloric restriction with the experimental diets shows that the diet containing L-carnitine alone and the diet containing the L-camitine and antioxidants mimic or behave most similar to caloric restriction in young mice. The data shown in tables 1 and 2 is plotted on the graphs represented in FIGS. 1 and 2. This graph shows the behavioral similarity of the target genes after caloric restriction and supplementation with L-carnitine and antioxidants in young mice. Thus, the data represents that administering carnitine and antioxidants to young mice has similar effects on gene expression to caloric restriction on young mice.

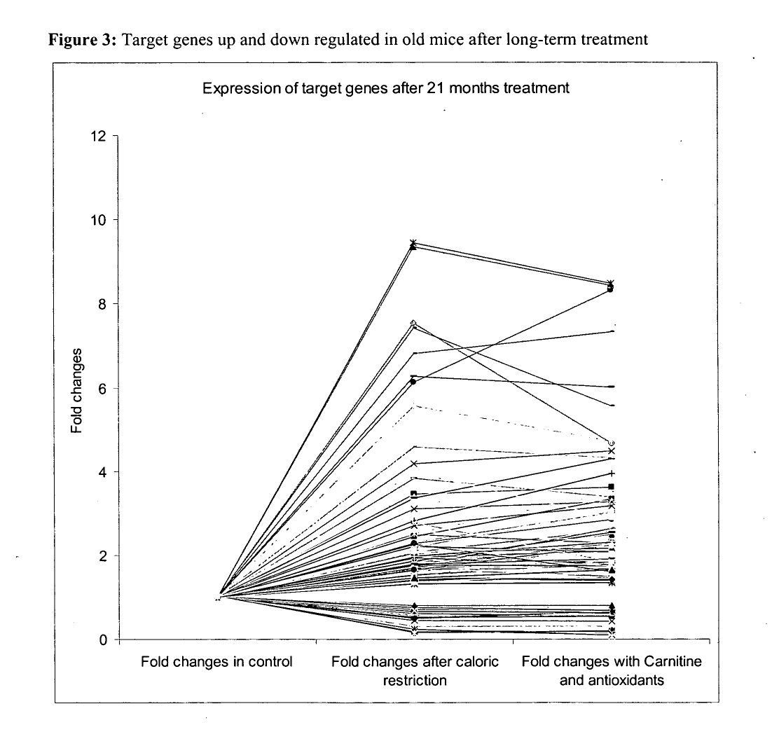

Table 3, below, illustrates the comparison of the effect of the diet containing carnitine and antioxidants with that of caloric restriction after long-term treatment (21 months). As shown in table 3, when the diets were administered for long treatment, the gene expression changes on the target genes are very similar for both diets. FIG. 3 graphically illustrates the data shown in table 3 and demonstrates the striking similarity of caloric restriction and supplementation with carnitine and antioxidant on gene expression, here expressed as fold changes over the control diet.

Accordingly, the data represented in the tables 1, 2 and 3 and FIGS. 1, 2 and 3 illustrate that the benefits of a caloric restriction can be obtained without the need for a subject to drastically reduce their calorie intake, and suffer from the many consequences of such a diet. A better alternative is surprisingly found by administrating a nutritional of a method for mimicking the effects of a caloric restricted diet on gene expression.

### EXAMPLES

The following examples are given by way of illustration only and in no way should be construed as limiting the subject matter of the present application. All percentages are given by weight unless otherwise indicated.

### Example 1

### Effect of Dietary Interventions with Antioxidants and Activators of Mitochondria Metabolism in a Murine Model by Gene Expression Profiling in Skeletal Muscle

Study Design:

Dietary intervention was of 3 months, all animal groups were fed Ad libitum except for the group of caloric restricted mice which as fed 67% of the daily food consumed by the control Ad libitum group. Animal weight was measured once a week.

The effect of short and long nutritional intervention was investigated. The short-term dietary interventions with diet A, B, C, D and F was initiated in young mice and lasted for three months. In a similar way, long-term interventions were initiated at three months of age for the following diets A and B and D and lasted twenty-one months.

Animals:

Male mice C57/B16 were obtained from Iffa credo (France) at 9 weeks of age. Upon arrival mice were housed by groups of 6 animals. After 3 weeks adaptation, mice (12 weeks old) were randomized 6 groups (A to F) of 12 mice each and housed individually. Dietary intervention was of 3 months; mice had free access to water and were submitted to 12 hours light and dark cycles.

Diets:

The control diet (diet A) composed of 18% proteins (soy and whey), 11% fat (soybean oil), 59% carbohydrates (starch+sucrose) and 10% cellulose was supplemented with either a cocktail of antioxidants comprising vitamin C, vitamin E, grape seed extract and cysteine (diet C) and/or L-camitine (diet D and F respectively). For caloric restriction (diet B) fat, starch and sucrose were reduced to provide 67% of the daily calorie consumption of the Ad-lib control group while providing 100% for proteins, minerals and vitamins. These diets are as follows:
Diet A—Control: 18% proteins (soy and whey), 11% fat, 59% carbohydrates, 5% cellulose.
Diet B—Caloric restriction: 18% proteins (soy and whey), 7.7% fat, 32.5% carbohydrates, 5% cellulose
Diet C—Cocktail of antioxidants : Diet A+0.19% vit C, 0.03% vit E, 0.075% grape seed extract, 0.4% cysteine.
Diet D: Carnitine and Antioxidants: Diet A+0.3% L- camitine+cocktail of antioxidants of diet C.
Diet F: Carnitine: Diet A+0.3% L- carnitine

RNA Preparation:

Mice were decapitated and dissected rapidly. Skeletal muscles (gastrocnemius) were immersed in RNAlatter (Ambion) and frozen at −80° C. until use. For RNA extraction, muscles were homogenized with ceramic beads (FastPrep, Q-Biogene) and the RNA extracted with Totally RNA kit (Ambion). The quality of the RNA was checked by Agilent technology. RNA pools from four mice each were created and hybridized to Affymetrix Murine U74Av2 high-density oligonucleotide microarrays.

Genomics:

The Global Error Assessment (GEA) methodology was used to select differentially expressed genes in the present invention. See Bioinformatics, Vol. 20, No. 16 (Oxford University Press 2004) pp. 2726-2737, the content of which is hereby incorporated by reference thereto. The goal was to select genes with statistically significant differential expression between treatments and ages of the mice of the study.

Results Obtained By Gene Profiling Analysis

As a first assessment, the five experimental diets were compared to the control diet and clustered (hierarchical clustering) using Spotfire. Differential gene expression profiles indicate that the diet containing both the carnitine and antioxidants modulates set of genes in a similar way to caloric restriction, as shown in Tables 1 to 2, and FIGS. 1 to 2.

### Example 2

### Dry Pet Food

A feed mixture is made up of about 58% by weight of corn, about 5.5% by weight of corn gluten, about 22% by weight of chicken meal, 2.5% dried chicory, 1% carnitine, and 1% creatine for stimulation of energy metabolism, 0.1% Vit C, vit E (150 IU/kg), 0.05% grape seed proanthocyanidin extract and 1% cysteine as antioxidant, salts, vitamins and minerals making up the remainder.

The fed mixture is fed into a preconditioner and moistened. The moistened feed is then fed into an extruder-cooker and gelatinized. The gelatinized matrix leaving the extruder is forced through a die and extruded. The extrudate is cut into pieces suitable for feeding to dogs, dried at about 110° C., for about 20 minutes, and cooled to form pellets.

This dry dog food is intended to improve or restore the age-related deficits in dogs.

### Example 3

### Dry Pet Food

A feed mixture is prepared as in example 1, using 2% carnitine for stimulation of energy metabolism and 0.05% ginkgo biloba extract as antioxidant. Then, the fed mixture is processed as in example 1. The dry dog food is also particularly intended to improve or restore the age-related deficits in dogs.

### Example 4

### Wet Canned Pet Food

A mixture is prepared from 73% of poultry carcass, pig lungs and beef liver (ground), 16% of wheat flour, 2% of dyes, vitamins, and inorganic salts, and 2% of carnitine for stimulation of energy metabolism and 0.4% green tea as antioxidant.

This mixture is emulsified at 12° C. and extruded in the form of a pudding which is then cooked at a temperature of 90° C. It is cooled to 30° C. and cut in chunks. 45% of these chunks are mixed with 55% of a sauce prepared from 98% of water, 1% of dye, and 1% of guar gum. Tinplate cans are filled and sterilized at 125° C. for 40 min.

### Example 5

### Wet Canned Pet Food

A mixture is prepared from 56% of poultry carcass, pig lungs and pig liver (ground), 13% of fish, 16% of wheat flour, 2% of plasma, 10.8% of water, 2.2% of dyes, 1% of semi refined kappa carrageenan, inorganic salts and 9% oil rich in monounsaturated fatty acids (olive oil) and 1% creatine for stimulation of energy metabolism and 1% taurine as antioxidant. This mixture is emulsified at 12° C. and extruded in the form of a pudding which is then cooked at a temperature of 90° C. It is cooled to 30° C. and cut in chunks.

30% of these chunks (having a water content of 58%) is incorporated in a base prepared from 23% of poultry carcass, 1% of guar gum, 1% of dye and aroma and 75% of water. Tinplate cans are then filled and sterilized at 127° C. for 60 min.

### Example 6

### Nutritional Formula

A nutritional composition is prepared, and which contains for 100 g of powder 15% of protein hydrolysate, 25% of fats, 55% carbohydrates (including 37% maltodextrin, 6% starch, and 12% sucrose), traces of vitamins and oligoelements to meet daily requirements, 2% minerals and 3% moisture and 2% pyruvate for stimulation of energy metabolism and 1% carnosine or carnosine precursor as antioxidant.

13 g of this powder is mixed in 100 ml of water. The obtained formula is particularly intended for reversing age-related gene expression alterations and restore or prevent age-related functional deficits in humans.

TABLE 1
Genes selected as significantly modulated by caloric restriction and
supplementation with L-carnitine and antioxidants in skeletal muscle of young mice after 3
months treatment (down regulation)

| | | | | | Fold | |
|---|---|---|---|---|---|---|
| | | | Fold | | Changes | |
| | | Fold | Changes | Fold | Carnitine | Fold |
| | | Changes | Caloric | Changes | and | Changes |
| Gene Title | Gene Symbol | Control | restriction | Antioxidants | antioxidants | Carnitine |
| zinc finger protein 289 | Zfp289 | 1 | 0.6 | 1.4 | 0.5 | 0.6 |
| zinc finger protein 101 | Zfp101 | 1 | 0.1 | 0.2 | 0.1 | 0.2 |
| exportin 7 | Xpo7 | 1 | 0.6 | 0.9 | 0.6 | 0.7 |
| vacuolar protein sorting 54 (yeast) | Vps54 | 1 | 0.5 | 0.6 | 0.6 | 0.4 |
| UPF3 regulator of nonsense transcripts homolog B (yeast) | Upf3b | 1 | 0.5 | 0.8 | 0.5 | 0.7 |
| ubiquitin-conjugating enzyme E2E 1, UBC4/5 homolog (yeast) | Ube2e1 | 1 | 0.6 | 0.5 | 0.6 | 0.7 |
| ubiquitin B | Ubb | 1 | 0.8 | 0.8 | 0.8 | 0.7 |
| tissue specific transplantation antigen P35B | Tsta3 | 1 | 0.6 | 1.2 | 0.4 | 0.9 |
| topoisomerase (DNA) I | Top1 | 1 | 0.6 | 0.6 | 0.5 | 0.5 |
| transportin 1 | Tnpo1 | 1 | 0.2 | 0.6 | 0.1 | 0.4 |
| transmembrane 4 superfamily member 3 | Tm4sf3 | 1 | 0.5 | 0.7 | 0.6 | 0.5 |
| transcription factor-like 1 | Tcfl1 | 1 | 0.6 | 0.9 | 0.7 | 0.7 |
| serine/threonine kinase receptor associated protein | Strap | 1 | 0.6 | 0.9 | 0.6 | 0.6 |
| sorbin and SH3 domain containing 1 | Sorbs1 | 1 | 0.2 | 0.2 | 0.2 | 0.8 |
| secretory leukocyte protease inhibitor | Slpi | 1 | 0.3 | 1.1 | 0.4 | 1.1 |
| solute carrier family 38, member 2 | Slc38a2 | 1 | 0.5 | 0.6 | 0.7 | 0.8 |
| solute carrier family 2 (facilitated glucose transporter), member 5 | Slc2a5 | 1 | 0.2 | 0.6 | 0.2 | 0.2 |
| S-phase kinase-associated protein 1A | Skp1a | 1 | 0.5 | 0.6 | 0.7 | 0.5 |
| serum/glucocorticoid regulated kinase | Sgk | 1 | 0.5 | 0.7 | 0.7 | 0.8 |
| secretory carrier membrane protein 2 | Scamp2 | 1 | 0.6 | 0.8 | 0.6 | 0.7 |
| SAR1a gene homolog 2 (S. cerevisiae) | Sara2 | 1 | 0.7 | 0.6 | 0.7 | 0.8 |
| RWD domain containing 1 | Rwdd1 | 1 | 0.5 | 1 | 0.7 | 0.6 |
| reticulon 4 | Rtn4 | 1 | 0.8 | 0.7 | 0.8 | 0.9 |
| Ras-related associated with diabetes | Rrad | 1 | 0.6 | 0.6 | 0.6 | 0.5 |
| ribonuclease P 14 kDa subunit (human) | Rpp14 | 1 | 0.6 | 0.8 | 0.6 | 0.7 |
| ribosomal protein L30 | Rpl30 | 1 | 0.8 | 0.8 | 0.8 | 0.7 |
| ribosomal protein L10A | Rpl10a | 1 | 0.4 | 0.8 | 0.6 | 0.4 |
| RNA binding motif protein 17 | Rbm17 | 1 | 0.6 | 0.9 | 0.6 | 0.7 |
| RAB2, member RAS oncogene family | Rab2 | 1 | 0.7 | 0.8 | 0.7 | 0.9 |
| proteasome (prosome, macropain) 26S subunit, non-ATPase, 8 | Psmd8 | 1 | 0.5 | 0.8 | 0.7 | 0.7 |
| proteasome (prosome, macropain) 26S subunit, non-ATPase, 11 | Psmd11 | 1 | 0.6 | 0.8 | 0.8 | 0.6 |
| protease (prosome, macropain) 26S subunit, ATPase 1 | Psmc1 | 1 | 0.2 | 1.3 | 0.2 | 0.5 |
| proteasome (prosome, macropain) subunit, alpha type 3 | Psma3 | 1 | 0.7 | 0.8 | 0.7 | 0.7 |
| PRP19/PSO4 homolog (S. cerevisiae) | Prp19 | 1 | 0.7 | 0.9 | 0.7 | 0.5 |
| polo-like kinase 1 (Drosophila) | Plk1 | 1 | 0.2 | 0.3 | 0.2 | 0.3 |
| pyruvate kinase, muscle | Pkm2 | 1 | 0.8 | 0.7 | 0.6 | 0.5 |
| polymeric immunoglobulin receptor | Pigr | 1 | 0.6 | 0.8 | 0.6 | 0.7 |
| pyruvate dehydrogenase kinase, isoenzyme 4 | Pdk4 | 1 | 0.3 | 0.7 | 0.6 | 0.9 |
| programmed cell death 4 | Pdcd4 | 1 | 0.4 | 0.7 | 0.4 | 0.6 |
| protocadherin alpha 12 | Pcdha12 | 1 | 0.6 | 0.6 | 0.3 | 0.4 |
| origin recognition complex, subunit 4-like (S. cerevisiae) | Orc4l | 1 | 0.6 | 0.7 | 0.6 | 0.6 |
| neuroblastoma ras oncogene | Nras | 1 | 0.6 | 0.8 | 0.6 | 0.6 |
| Niemann Pick type C1 | Npc1 | 1 | 0.6 | 0.7 | 0.7 | 0.6 |
| neurogenic differentiation 6 | Neurod6 | 1 | 0.1 | 0.2 | 0.1 | 0.1 |
| myosin Va | Myo5a | 1 | 0.1 | 0.7 | 0.1 | 0.1 |
| mucin 1, transmembrane | Muc1 | 1 | 0.3 | 0.3 | 0.3 | 0.5 |
| metallothionein 2 | Mt2 | 1 | 0.3 | 0.3 | 0.3 | 0.3 |
| metallothionein 1 | Mt1 | 1 | 0.6 | 0.8 | 0.6 | 0.5 |
| max binding protein | Mnt | 1 | 0.2 | 1.1 | 0.3 | 0.6 |
| matrin 3 | Matr3 | 1 | 0.6 | 0.8 | 0.6 | 0.6 |
| microtubule-associated protein tau | Mapt | 1 | 0.6 | 0.7 | 0.6 | 0.5 |
| microtubule-associated protein, RP/EB family, member 3 | Mapre3 | 1 | 0.4 | 0.9 | 0.5 | 0.4 |
| microtubule-actin crosslinking factor 1 | Macf1 | 1 | 0.5 | 0.8 | 0.5 | 0.5 |
| lipopolysaccharide binding protein | Lbp | 1 | 0.5 | 0.8 | 0.5 | 0.6 |
| keratin associated protein 3-1 | Krtap3-1 | 1 | 0.5 | 1.2 | 0.3 | 0.7 |
| killer cell lectin-like receptor subfamily B member 1C | Klrb1c | 1 | 0.3 | 0.6 | 0.2 | 0.2 |
| Jun-B oncogene | Junb | 1 | 0.4 | 0.3 | 0.3 | 0.4 |
| inositol 1,4,5-triphosphate receptor 1 | Itpr1 | 1 | 0.3 | 0.7 | 0.5 | 0.4 |
| integrin beta 1 (fibronectin receptor beta) | Itgb1 | 1 | 0.7 | 0.7 | 0.7 | 0.7 |
| integrin alpha V | Itgav | 1 | 0.3 | 0.5 | 0.3 | 0.2 |
| integrin alpha V | Itgav | 1 | 0.7 | 0.9 | 0.7 | 0.7 |
| insulin-like growth factor binding protein 5 | Igfbp5 | 1 | 0.6 | 0.7 | 0.7 | 0.6 |
| homeo box D8 | Hoxd8 | 1 | 0.5 | 0.8 | 0.7 | 0.6 |
| 3-hydroxy-3-methylglutaryl-Coenzyme A lyase | Hmgcl | 1 | 0.5 | 0.7 | 0.5 | 0.5 |
| histone deacetylase 2 | Hdac2 | 1 | 0.6 | 0.6 | 0.5 | 0.8 |
| glutathione S-transferase, mu 5 | Gstm5 | 1 | 0.6 | 0.9 | 0.8 | 0.6 |
| glutathione peroxidase 7 | Gpx7 | 1 | 0.1 | 0.4 | 0.2 | 0.3 |
| G-protein coupled receptor 12 | Gpr12 | 1 | 0.5 | 0.2 | 0.6 | 0.7 |
| growth arrest specific 5 | Gas5 | 1 | 0.4 | 0.8 | 0.6 | 0.6 |
| UDP-N-acetyl-alpha-D-galactosamine:polypeptide | Galnt1 | 1 | 0.3 | 0.7 | 0.5 | 0.5 |
| N-acetylgalactosaminyltra | | | | | | |
| Fc receptor, IgG, low affinity Ilb | Fcgr2b | 1 | 0.2 | 0.2 | 0.1 | 0.1 |
| epidermal growth factor receptor pathway substrate 15 | Eps15 | 1 | 0.6 | 0.8 | 0.7 | 0.7 |
| glutamyl aminopeptidase | Enpep | 1 | 0.6 | 0.7 | 0.6 | 0.6 |
| eukaryotic translation initiation factor 4E binding protein 2 | Eif4ebp2 | 1 | 0.5 | 0.9 | 0.6 | 0.6 |
| eukaryotic translation initiation factor 4E binding protein 1 | Eif4ebp1 | 1 | 0.5 | 0.8 | 0.7 | 0.6 |
| eukaryotic translation initiation factor 2, subunit 3, structural gene | Eif2s3x | 1 | 0.7 | 0.9 | 0.7 | 0.7 |
| X-linked | | | | | | |
| differentially expressed in B16F10 1 | Deb1 | 1 | 0.7 | 1 | 0.6 | 0.8 |
| damage specific DNA binding protein 1 | Ddb1 | 1 | 0.6 | 0.9 | 0.7 | 0.7 |
| DNA segment, Chr 8, ERATO Doi 69, expressed | D8Ertd69e | 1 | 0.1 | 0.2 | 0 | 1.2 |
| coatomer protein complex, subunit beta 2 (beta prime) | Copb2 | 1 | 0.5 | 0.7 | 0.5 | 0.7 |
| CCAAT/enhancer binding protein (C/EBP), delta | Cebpd | 1 | 0.5 | 0.7 | 0.4 | 0.5 |
| cadherin 10 | Cdh10 | 1 | 0.1 | 0.2 | 0.1 | 0.4 |
| CD164 antigen | Cd164 | 1 | 0.5 | 0.8 | 0.7 | 0.8 |
| chemokine (C-C motif) ligand 9 | Ccl9 | 1 | 0.5 | 0.6 | 0.5 | 0.5 |
| core binding factor beta | Cbfb | 1 | 0.7 | 0.9 | 0.7 | 0.7 |
| capping protein (actin filament) muscle Z-line, beta | Capzb | 1 | 0.6 | 1.1 | 0.6 | 0.6 |
| capping protein (actin filament) muscle Z-line, alpha 2 | Capza2 | 1 | 0.6 | 0.7 | 0.7 | 0.7 |
| complement component 4 (within H-2S) | C4 | 1 | 0.5 | 0.5 | 0.6 | 0.6 |
| ATPase type 13A | Atp13a | 1 | 0.4 | 0.7 | 0.3 | 0.7 |
| actin related protein 2/3 complex, subunit 2 | Arpc2 | 1 | 0.5 | 0.9 | 0.7 | 0.6 |
| AT rich interactive domain 1A (Swi1 like) | Arid1a | 1 | 0.7 | 0.7 | 0.7 | 0.6 |
| apolipoprotein B editing complex 2 | Apobec2 | 1 | 0.4 | 0.8 | 0.5 | 0.5 |
| acidic (leucine-rich) nuclear phosphoprotein 32 family, | Anp32e | 1 | 0.5 | 0.7 | 0.7 | 0.6 |
| member E | | | | | | |
| RIKEN cDNA 6330407G11 gene | 6330407G11Rik | 1 | 0.3 | 0.3 | 0.3 | 0.6 |
| RIKEN cDNA 5730497N03 gene | 5730497N03Rik | 1 | 0.1 | 0.5 | 0.1 | 0.2 |
| RIKEN cDNA 5730454B08 gene | 5730454B08Rik | 1 | 0.7 | 0.7 | 0.7 | 0.8 |
| RIKEN cDNA 2700059D21 gene | 2700059D21Rik | 1 | 0.7 | 1 | 0.6 | 0.5 |
| RIKEN cDNA 2610034N03 gene | 2610034N03Rik | 1 | 0.5 | 0.8 | 0.6 | 0.5 |
| RIKEN cDNA 2310073E15 gene | 2310073E15Rik | 1 | 0.2 | 0.2 | 0.2 | 1.2 |
| RIKEN cDNA 2310016A09 gene | 2310016A09Rik | 1 | 0.6 | 1 | 0.7 | 0.7 |
| RIKEN cDNA 2210419D22 gene | 2210419D22Rik | 1 | 0.5 | 0.9 | 0.6 | 0.8 |
| RIKEN cDNA 2010012F05 gene | 2010012F05Rik | 1 | 0.7 | 0.7 | 0.7 | 0.8 |
| RIKEN cDNA 0610013E23 gene | 0610013E23Rik | 1 | 0.5 | 0.7 | 0.5 | 0.6 |
| Transcribed sequence with strong similarity to protein sp: | — | 1 | 0.7 | 0.9 | 0.8 | 0.8 |
| P49840 (H. sapiens | | | | | | |
| Transcribed sequences | — | 1 | 0.7 | 0.9 | 0.7 | 0.7 |

TABLE 2
Genes selected as significantly modulated by caloric restriction and
supplementation with L-carnitine and antioxidants in skeletal muscle of young mice after 3
months treatments (up regulation)

| | | | | | Fold | Fold |
|---|---|---|---|---|---|---|
| | | | Fold | | Changes | Chang- |
| | | Fold | Changes | Fold | Carnitine | es |
| | | Changes | Caloric | Changes | and | Carni- |
| Gene Title | Gene Symbol | Control | restriction | Antioxidants | antioxidants | tine |
| histone 2, H3c2 | Hist2h3c2 | 1 | 1.6 | 1.3 | 1.7 | 1.5 |
| NADH dehydrogenase (ubiquinone) 1 beta subcomplex 3 | Ndufb3 | 1 | 1.3 | 1 | 1.5 | 1.2 |
| AKT1 substrate 1 (proline-rich) | Akt1s1 | 1 | 1.6 | 1.1 | 1.7 | 1.4 |
| sorcin | Sri | 1 | 3.2 | 2.6 | 2.6 | 2.3 |
| aldehyde dehydrogenase 2, mitochondrial | Aldh2 | 1 | 2.3 | 0.9 | 1.7 | 1.7 |
| suppression of tumorigenicity 13 | St13 | 1 | 2.3 | 1.2 | 2.1 | 1.5 |
| CD97 antigen | Cd97 | 1 | 7.5 | 1.4 | 6.4 | 5.4 |
| sepiapterin reductase | Spr | 1 | 1.8 | 1.6 | 1.7 | 1.4 |
| RIKEN cDNA 3110038L01 gene | 3110038L01Rik | 1 | 1.7 | 1.1 | 1.9 | 1.4 |
| sorting nexin 2 | Snx2 | 1 | 13.1 | 2.8 | 12.5 | 1.5 |
| G0/G1 switch gene 2 | G0s2 | 1 | 4.5 | 2.3 | 2.5 | 1.5 |
| endogenous retroviral sequence 4 (with leucine t-RNA primer) | Erv4 | 1 | 4.8 | 0.9 | 9.4 | 5.2 |
| tryptophanyl-tRNA synthetase | Wars | 1 | 2.6 | 1.4 | 2.1 | 1.9 |
| pericentrin 2 | Pcnt2 | 1 | 4.3 | 3.1 | 5.7 | 1.6 |
| ras homolog gene family, member A | Rhoa | 1 | 1.5 | 1.1 | 1.6 | 1.4 |
| actin, beta, cytoplasmic | Actb | 1 | 1.5 | 1.2 | 1.4 | 1.4 |
| lactate dehydrogenase 2, B chain | Ldh2 | 1 | 2.4 | 1.7 | 2.4 | 1.6 |
| resistin | Retn | 1 | 4.5 | 2.7 | 5.2 | 1.9 |
| carbonic anhydrase 4 | Car4 | 1 | 3 | 1.5 | 2.6 | 2.3 |
| nicotinamide nucleotide transhydrogenase | Nnt | 1 | 2.1 | 1.4 | 1.8 | 1.7 |
| tubulin, gamma 2 | Tubg2 | 1 | 2.1 | 1.5 | 2.6 | 1.6 |
| Kruppel-like factor 3 (basic) | Klf3 | 1 | 8.1 | 3.2 | 7.5 | 5.9 |
| mitogen activated protein kinase 9 | Mapk9 | 1 | 3.8 | 1.1 | 2.9 | 2.8 |
| hemoglobin, beta adult major chain | Hbb-b1 | 1 | 2 | 1.4 | 1.3 | 1.2 |
| RIKEN cDNA 6720463E02 gene | 6720463E02Rik | 1 | 1.6 | 0.8 | 1.5 | 1.3 |
| lysophospholipase 1 | Lypla1 | 1 | 4.7 | 2.7 | 5.8 | 4.8 |
| tenascin XB | Tnxb | 1 | 2.1 | 0.7 | 1.5 | 1.4 |
| chemokine (C motif) ligand 1 | Xcl1 | 1 | 5.9 | 2.4 | 5.9 | 4.7 |
| signal transducing adaptor molecule (SH3 domain and ITAM motif) 2 | Stam2 | 1 | 6 | 1 | 7 | 4.3 |
| DNA segment, Chr 19, ERATO Doi 678, expressed | D19Ertd678e | 1 | 5 | 0.9 | 9.5 | 5.2 |
| dual-specificity tyrosine-(Y)-phosphorylation regulated kinase 1a | Dyrk1a | 1 | 2.5 | 1.2 | 2.7 | 1.8 |
| utrophin | Utrn | 1 | 2.6 | 1.1 | 2.6 | 1.7 |
| kinase suppressor of ras | Ksr | 1 | 2.1 | 1.1 | 2.2 | 1.8 |
| baculoviral IAP repeat-containing 4 | Birc4 | 1 | 8.8 | 1.1 | 8 | 5.9 |
| calcium/calmodulin-dependent protein kinase II alpha | Camk2a | 1 | 5.6 | 1.7 | 7.3 | 4.6 |
| ATPase, Na+/K+ transporting, beta 2 polypeptide | Atp1b2 | 1 | 3.9 | 2.1 | 5.7 | 4.1 |
| — | — | 1 | 1.8 | 1.5 | 2 | 1.8 |
| estrogen receptor 1 (alpha) | Esr1 | 1 | 11.2 | 2.7 | 14.2 | 6.5 |
| transglutaminase 3, E polypeptide | Tgm3 | 1 | 6.3 | 1.5 | 4.5 | 2.5 |
| sialyltransferase 6 (N-acetyllacosaminide alpha 2,3-sialyltransferase) | Siat6 | 1 | 2.2 | 1.5 | 3 | 2 |
| integrin alpha V | Itgav | 1 | 5.3 | 2.2 | 5.2 | 4.6 |
| prosaposin | Psap | 1 | 26.6 | 2.1 | 19.3 | 17.3 |
| ribosomal protein L10A | Rpl10a | 1 | 1.4 | 1.1 | 1.4 | 1.2 |
| 5-hydroxytryptamine (serotonin) receptor 1A | Htr1a | 1 | 5 | 2.3 | 5.5 | 4.2 |
| lipoprotein lipase | Lpl | 1 | 1.6 | 1.4 | 1.7 | 1.4 |
| carboxypeptidase D | Cpd | 1 | 2 | 1.3 | 2.3 | 1.9 |
| RIKEN cDNA 4732477C12 gene | 4732477C12Rik | 1 | 1.7 | 1.4 | 2.1 | 1.2 |
| RIKEN cDNA E030006K04 gene | E030006K04Rik | 1 | 7.5 | 3 | 11.1 | 2.9 |
| — | — | 1 | 1.6 | 1 | 1.5 | 1.1 |

TABLE 3

| | | | | |
|---|---|---|---|---|
| | | | Fold Changes | Fold Changes |
| | | Fold Changes | Caloric | Carnitine and |
| Gene Title | Gene Symbol | Control | restriction | antioxidants |

Genes selected as significantly modulated by caloric restriction and
supplementation with L-carnitine and antioxidants in skeletal muscle of old mice after 21
months treatment treatments (up and down regulation)

| | | | | |
|---|---|---|---|---|
| huntingtin interacting protein 2 | Hip2 | 1 | 1.7 | 1.7 |
| ribosomal protein S13 | Rps13 | 1 | 3.5 | 3.6 |
| pyruvate carboxylase | Pcx | 1 | 3.4 | 2.5 |
| tissue inhibitor of metalloproteinase 2 | Timp2 | 1 | 2.1 | 1.8 |
| troponin I, skeletal, fast 2 | Tnni2 | 1 | 1.4 | 1.4 |
| ATP synthase, H+ transporting, mitochondrial F1 complex, epsilon subunit | Atp5e | 1 | 1.7 | 1.5 |
| zinc finger protein 265 | Zfp265 | 1 | 0.6 | 0.5 |
| ATPase, Na+/K+ transporting, alpha 1 polypeptide | Atp1a1 | 1 | 1.7 | 2.6 |
| golgi SNAP receptor complex member 2 | Gosr2 | 1 | 0.2 | 0.1 |
| fatty acid binding protein 3, muscle and heart | Fabp3 | 1 | 1.3 | 1.6 |
| ribosomal protein L29 | Rpl29 | 1 | 0.2 | 0.1 |
| serine/threonine kinase receptor associated protein | Strap | 1 | 0.6 | 0.6 |
| guanine nucleotide binding protein, alpha inhibiting 3 | Gnai3 | 1 | 1.7 | 1.9 |
| haptoglobin | Hp | 1 | 1.8 | 1.5 |
| Similar to hypothetical protein FLJ11749 (LOC208092), mRNA | — | 1 | 2.1 | 2.1 |
| RIKEN cDNA 1500003O03 gene | 1500003O03Rik | 1 | 1.7 | 2.0 |
| eukaryotic translation initiation factor 3, subunit 1 alpha | Eif3s1 | 1 | 0.7 | 0.6 |
| RIKEN cDNA 1700037H04 gene | 1700037H04Rik | 1 | 3.8 | 3.4 |
| RIKEN cDNA B830022L21 gene | B830022L21Rik | 1 | 0.3 | 0.3 |
| topoisomerase (DNA) I | Top1 | 1 | 0.5 | 0.6 |
| cold shock domain protein A | Csda | 1 | 0.7 | 0.7 |
| ribosomal protein S27 | Rps27 | 1 | 1.7 | 1.5 |
| Similar to 60S ribosomal protein L34 (LOC384425), mRNA | — | 1 | 2.1 | 2.6 |
| core promoter element binding protein | Copeb | 1 | 0.5 | 0.6 |
| capping protein (actin filament) muscle Z-line, alpha 2 | Capza2 | 1 | 0.7 | 0.6 |
| sarcolemma associated protein | Slmap | 1 | 0.7 | 0.7 |
| signal transducer and activator of transcription 3 | Stat3 | 1 | 1.5 | 1.8 |
| CD24a antigen | Cd24a | 1 | 0.8 | 0.7 |
| lysozyme | Lyzs | 1 | 0.5 | 0.5 |
| insulin-like growth factor binding protein 4 | Igfbp4 | 1 | 1.8 | 1.9 |
| procollagen, type IX, alpha 3 | Col9a3 | 1 | 4.2 | 4.5 |
| Jun proto-oncogene related gene d1 | Jund1 | 1 | 1.3 | 1.3 |
| upstream transcription factor 2 | Usf2 | 1 | 1.6 | 1.7 |
| Myb protein P42POP | P42pop | 1 | 1.4 | 1.5 |
| phosphodiesterase 4B, cAMP specific | Pde4b | 1 | 2.3 | 3.0 |
| RIKEN cDNA 2210409E12 gene | 2210409E12Rik | 1 | 3.4 | 4.3 |
| Similar to corneodesmosin precursor | — | 1 | 2.0 | 2.3 |
| phosphatidylinositol glycan, class T | Pigt | 1 | 1.5 | 1.9 |
| regulator of G-protein signaling 5 | Rgs5 | 1 | 2.1 | 2.4 |
| T-cell receptor alpha chain | Tcra | 1 | 2.7 | 3.2 |
| adenylosuccinate lyase | Adsl | 1 | 0.7 | 0.7 |
| E74-like factor 3 | Elf3 | 1 | 2.2 | 2.1 |
| small proline-rich protein 1B | Sprr1b | 1 | 1.4 | 1.4 |
| mitogen activated protein kinase 9 | Mapk9 | 1 | 1.9 | 1.8 |
| phosphoglycerate kinase 2 | Pgk2 | 1 | 6.3 | 6.0 |
| perlecan (heparan sulfate proteoglycan 2) | Hspg2 | 1 | 1.8 | 1.9 |
| zinc finger protein 106 | Zfp106 | 1 | 1.7 | 2.3 |
| SET and MYND domain containing 1 | Smyd1 | 1 | 1.9 | 1.8 |
| protein kinase, cAMP dependent regulatory, type I beta | Prkar1b | 1 | 2.5 | 2.3 |
| RAB10, member RAS oncogene family | Rab10 | 1 | 1.6 | 1.7 |
| lysophospholipase 1 | Lypla1 | 1 | 1.6 | 1.9 |
| lysophospholipase 1 | Lypla1 | 1 | 1.7 | 1.8 |
| phosphotidylinositol transfer protein, beta | Pitpnb | 1 | 4.6 | 4.3 |
| chemokine (C—C motif) ligand 2 | Ccl2 | 1 | 0.2 | 0.1 |
| RIKEN cDNA 5430432P15 gene | 5430432P15Rik | 1 | 0.7 | 0.6 |
| acid phosphatase, prostate | Acpp | 1 | 2.4 | 3.3 |
| homeodomain interacting protein kinase 3 | Hipk3 | 1 | 1.9 | 2.5 |
| regulator of G-protein signaling 5 | Rgs5 | 1 | 2.2 | 1.8 |
| CD4 antigen | Cd4 | 1 | 2.5 | 3.5 |
| eukaryotic translation initiation factor 4, gamma 1 | Eif4g1 | 1 | 2.3 | 2.8 |
| epimorphin | Epim | 1 | 2.0 | 2.1 |
| signal transducer and activator of transcription 5B | Stat5b | 1 | 1.7 | 2.3 |
| retinoid X receptor alpha | Rxra | 1 | 1.9 | 2.5 |
| solute carrier family 2 (facilitated glucose transporter), member 3 | Slc2a3 | 1 | 7.5 | 4.7 |
| chemokine (C—C motif) ligand 8 | Ccl8 | 1 | 0.2 | 0.3 |
| acidic (leucine-rich) nuclear phosphoprotein 32 family, member A | Anp32a | 1 | 7.8 | 10.7 |
| Transcribed sequence with weak similarity to protein ref: NP_115973.1 (H. [symbol] | — | 1 | 0.4 | 0.4 |
| rho/rac guanine nucleotide exchange factor (GEF) 2 | Arhgef2 | 1 | 1.8 | 1.8 |
| ATPase, Na+/K+ transporting, beta 2 polypeptide | Atp1b2 | 1 | 1.7 | 1.8 |
| src homology 2 domain-containing transforming protein C1 | Shc1 | 1 | 5.6 | 4.7 |

Genes selected as significantly modulated by caloric restriction and
supplementation with L-carnitine and antioxidants in skeletal muscle of old mice after 21
months treatment

| | | | | |
|---|---|---|---|---|
| DnaJ (Hsp40) homolog, subfamily B, member 4 | Dnajb4 | 1 | 1.7 | 1.7 |
| — | — | 1 | 1.7 | 1.8 |
| HLA-B-associated transcript 3 | Bat3 | 1 | 1.5 | 2.3 |
| expressed sequence AA408556 | AA408556 | 1 | 1.6 | 1.8 |
| myelin transcription factor 1-like | Myt1l | 1 | 3.3 | 3.3 |
| protein kinase, cAMP dependent regulatory, type II alpha | Prkar2a | 1 | 1.8 | 2.3 |
| ATP-binding cassette, sub-family C (CFTR/MRP), member 9 | Abcc9 | 1 | 2.0 | 1.8 |
| killer cell lectin-like receptor, subfamily A, member 7 | Kira7 | 1 | 6.1 | 8.3 |
| potassium inwardly rectifying channel, subfamily J, member 11 | Kcnj11 | 1 | 1.7 | 2.2 |
| T-box 14 | Tbx14 | 1 | 1.7 | 1.7 |
| — | — | 1 | 1.5 | 1.5 |
| DnaJ (Hsp40) homolog, subfamily B, member 5 | Dnajb5 | 1 | 2.0 | 2.3 |
| expressed sequence AA407151 | AA407151 | 1 | 0.7 | 0.6 |
| RIKEN cDNA 2900097C17 gene | 2900097C17Rik | 1 | 2.1 | 2.2 |
| vav 1 oncogene | Vav1 | 1 | 3.1 | 3.3 |
| serine (or cysteine) proteinase inhibitor, clade B, member 8 | Serpinb8 | 1 | 0.2 | 0.2 |
| chemokine (C-X-C motif) ligand 2 | Cxcl2 | 1 | 0.2 | 0.2 |
| — | — | 1 | 2.0 | 1.7 |
| expressed sequence AA675035 | AA675035 | 1 | 6.8 | 7.3 |
| — | — | 1 | 0.6 | 0.6 |
| RAB10, member RAS oncogene family | Rab10 | 1 | 1.5 | 1.4 |
| MAP kinase-activated protein kinase 2 | Mapkapk2 | 1 | 2.1 | 2.0 |
| carbonic anhydrase 3 | Car3 | 1 | 0.8 | 0.8 |
| homeo box D8 | Hoxd8 | 1 | 0.7 | 0.6 |
| regulator of G-protein signalling 10 | Rgs10 | 1 | 0.2 | 0.1 |
| sema domain, transmembrane domain (TM), and cytoplasmic domain, (se [symbol] | Sema6c | 1 | 1.6 | 2.2 |
| Cbp/p300-interacting transactivator with Glu/Asp-rich carboxy-terminal dom [symbol] | Cited1 | 1 | 2.8 | 3.9 |
| mannose-P-dolichol utilization defect 1 | Mpdu1 | 1 | 7.4 | 5.6 |
| protein phosphatase 6, catalytic subunit | Ppp6c | 1 | 1.8 | 1.9 |
| scleraxis | Scx | 1 | 0.5 | 0.7 |
| phosphorylase kinase, gamma 2 (testis) | Phkg2 | 1 | 4.0 | 2.9 |
| mitogen-activated protein kinase kinase kinase kinase 4 | Map4k4 | 1 | 9.3 | 8.4 |
| dihydroorotate dehydrogenase | Dhodh | 1 | 2.8 | 1.8 |
| adipose differentiation related protein | Adfp | 1 | 9.4 | 8.5 |
| zinc finger protein 94 | Zfp94 | 1 | 1.3 | 1.6 |
| acyl-Coenzyme A dehydrogenase, short chain | Acads | 1 | 2.6 | 2.3 |
| RNA binding motif protein 9 | Rbm9 | 1 | 2.2 | 1.6 |
| — | — | 1 | 2.2 | 3.0 |
| glucosamine (N-acetyl)-6-sulfatase | Gns | 1 | 0.1 | 0.2 |
| — | — | 1 | 1.4 | 1.6 |
| prolyl 4-hydroxylase, beta polypeptide | P4hb | 1 | 1.5 | 1.6 |
| selenium binding protein 1 | Selenbp1 | 1 | 0.5 | 0.4 |
| RIKEN cDNA 1500003O03 gene | 1500003O03Rik | 1 | 0.1 | 0.1 |
| ferredoxin reductase | Fdxr | 1 | 2.1 | 2.2 |

1. A method for delaying mitochondria dysfunction occurring in a mammal during aging, which method comprises administering to a mammal in need of or desirous of such treatment a combination that is able to mimic the effects of caloric restriction on gene expression, the combination containing (a) a carnitine compound, and (b) at least one antioxidant in an amount effective to reduce or prevent oxidative damage resulting from disruption of ATP/ADP or NAD+/NADH homeostasis due to increased substrate availability or utilization in aged mitochondria, and being administered in an amount effective to modulate or regulate expression of genes linked to energy metabolism.

2. The method of claim 1, wherein the method includes modulating gene expression of a target gene without restricting caloric intake to increase longevity.

3. The method of claim 1, wherein the target gene is involved in energy production, mitochondria biogenesis, proteases, or free radical production, free radical detoxification or modulators of inflammation, or apoptosis.

4. The method of claim 1, wherein the method reverses or retards oxidative damage to mitochondria.

5. The method of claim 1, wherein the camitine compound is L-camitine, and further wherein the L-carnitine is administered in an amount of at least 1 mg per kg of body weight per day.

6. The method of claim 1, wherein the antioxidant is one or more of thiol, lipoic acid, cysteine, cystine, methionine, S-adenosyl-methionine, taurine, glutathione, vitamin C, vitamin E, tocopherols and tocotrienols, carotenoids, carotenes, lycopene, lutein, zeaxanthine, ubiquinones, tea catechins, coffee extracts, ginkgo biloba extracts, grape or grape seed extracts, spice extracts, soy extracts, containing isoflavones, phytoestrogens ursodeoxycholic acid, ursolic acid, ginseng, or gingenosides, and further wherein the antioxidant is administered in an amount of at least 0.025 mg per kg of body weight per day.

7. The method of claim 1, wherein the camitine compound and the antioxidant is in combination with a molecule that stimulates metabolism selected from the group consisting of creatine, omega-3 fatty acids, cardiolipin, nicotinamide, or carbohydrate.

8. The method of claim 1, wherein the carnitine and the antioxidant is administered to the mammal in a food substrate.

9. The method of claim 8, wherein the food substrate is a nutritionally complete food substrate or a food supplement.

10. The method of claim 9, wherein the nutritionally complete food substrate is a pet food.

11. The method of claim 1 wherein the combination further comprises a molecule that stimulates energy metabolism.

12. The method of claim 11, wherein the target gene is one which is involved in energy production, mitochondria biogenesis, proteases, or free radical production, free radical detoxification or modulators of inflammation, apoptosis.

13. The method of claim 11, wherein the molecule that stimulates energy metabolism is creatine, fatty acids, cardiolipin nicotinamide, carbohydrate or any combination thereof.

14. The method of claim 11, wherein the molecule that stimulates energy metabolism is administered in an amount of at least 1 mg per kg of body weight per day.

15. The method of claim 11, wherein the antioxidant is one or more of thiol, lipoic acid, cysteine, cystine, methionine, S-adenosyl-methionine, taurine, glutathione, vitamin C, vitamin E, tocopherols and tocotrienols, carotenoids, carotenes, lycopene, lutein, zeaxanthine, ubiquinones, tea catechins, coffee extracts, ginkgo biloba extracts, grape or grape seed extracts, spice extracts, soy extracts, containing isoflavones, phytoestrogens ursodeoxycholic acid, ursolic acid, ginseng, or gingenosides, and further wherein the antioxidant is administered in an amount of at least 0.025 mg per kg of body weight per day.

16. The method of claim 11, wherein the carnitine, antioxidant, and molecule that stimulates energy metabolism is administered to the mammal in a food substrate.

17. The method of claim 11, wherein the method improves mitochondrial function and retards or reverses age associated oxidative damage to the mitochondria.

18. The method of claim 11, wherein the gene expression of a target gene is modulated such that the modulated gene expression mimics an effect of caloric restriction without a need for reducing caloric intake.

19. The method of claim 11, wherein the method improves at least one of skeletal and cardiac muscle function, vascular function, cognitive function, vision, hearing olfaction, skin and coat quality, bone and joint health, renal health, digestion, immune function, insulin sensitivity, inflammatory processes, and longevity in mammals.

20. A pet food composition that includes carnitine and at least one antioxidant, wherein the food composition is capable of mimicking an effect of caloric restriction on gene expression of a target gene.
